(19) United States
(12) Patent Application Publication
(10) Pub. No.: US 2023/0404922 A1
(43) Pub. Date: Dec. 21, 2023

(54) MICROSPHERE FORMULATIONS COMPRISING BTK INHIBITORS AND METHODS FOR MAKING AND USING THE SAME

(71) Applicant: Oakwood Laboratories, LLC, Oakwood Village, OH (US)

(72) Inventors: Michaela Giltner, Akron, OH (US); Rachel Galaska, Willoughby, OH (US); Tracy Richey, Stow, OH (US); Mark Smith, Venetia, PA (US)

(21) Appl. No.: 18/459,831

(22) Filed: Sep. 1, 2023

Related U.S. Application Data
(63) Continuation-in-part of application No. PCT/US2022/070910, filed on Mar. 2, 2022.
(60) Provisional application No. 63/156,020, filed on Mar. 3, 2021.
(60) Provisional application No. 63/511,923, filed on Jul. 5, 2023.

(51) Int. Cl. A61K 9/16; A61K 31/519

(52) U.S. Cl. CPC A61K 9/1647; A61K 31/519; A61K 9/0019

(57) ABSTRACT

Extended-release microsphere formulations comprising a BTK inhibitor are provided. In one aspect, the microsphere formulations are characterized in that the BTK inhibitor is ibrutinib, and the ibrutinib is released in vivo in humans over a period of from about 7 to about 28 days. Methods for making and using the formulations are also provided.

### CROSS-REFERENCE TO RELATED APPLICATIONS

This application is a continuation in part of International Application No. PCT/US2022/070910, filed on Mar. 2, 2022, which claims the benefit of U.S. Provisional Application No. 63/156,020, filed on Mar. 3, 2021. This application also claims the benefit of U.S. Provisional Application No. 63/511,923, filed on Jul. 5, 2023. Each of these applications is incorporated by reference herein in its entirety.

### BACKGROUND

B-cells account for up to 25% of all cells in some cancers. By inhibiting the Bruton's Tyrosine Kinase (“BTK”) enzyme involved in B-cell receptor signaling, BTK inhibitors cause detachment of malignant B-cells from cancer sites into blood, which results in cell death. BTK inhibition reduces the proliferation of malignant B-cells and decreases the survival of malignant B-cells.

Ibrutinib (chemical formula C₂₅H₂₄N₆O₂; CAS Number 936563-96-1), characterized by the general structure:

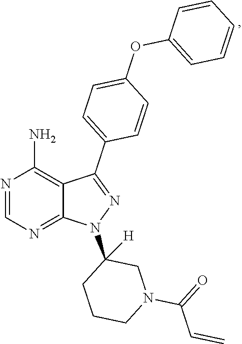 [SMILES: C=CC(=O)N1CCC[C@@H](n2nc(-c3ccc(Oc4ccccc4)cc3)c3c(N)ncnc32)C1]

is a BTK inhibitor.

Ibrutinib, alone and in combination with other drugs, has been approved by the U.S. Food and Drug Administration (the “FDA”) for the treatment of mantle cell lymphoma (“MCL”), chronic lymphocytic leukemia (“CLL”), Waldenstrom's macroglobulinemia, small lymphocytic lymphoma (“SLL”), relapsed/refractory marginal zone lymphoma in patients who require systemic therapy and have received at least one prior anti-CD20-based therapy, and graft-versus-host disease, among other diseases.

Two other BTK inhibitors have been approved by the FDA: acalabrutinib (approved for treatment of relapsed MCL) and zanubrutinib (approved for treatment of MCL). Several other drugs that inhibit BTK are in clinical trials, including evobrutinib for multiple sclerosis; ABBV-105 for systemic lupus erythematosus; fenebrutinib for rheumatoid arthritis, systemic lupus erythematosus, and chronic spontaneous urticaria; GS-4059 for non-Hodgkin's lymphoma and/or CLL; Spebrutinib (AVL-292, CC-292); and HM71224 for autoimmune diseases.

All of the currently approved BTK inhibitors are oral formulations. Oral formulations may have several disadvantages. For example, oral formulations may require closely timed, successive dosages under the supervision of a physician. Further, some BTK inhibitors may have low and variable oral bioavailability. For example, ibrutinib may have an oral bioavailability of only 2.9% in the fasted state, but this can vary from patient to patient.

A need exists for a highly bioavailable formulation comprising ibrutinib that may be administered by a long-acting, sustained release injection, without the need for patients to administer closely timed, successive dosages under supervision from their physician.

### SUMMARY

Microsphere formulations comprising ibrutinib are provided. The microsphere formulations comprise polymer microspheres, each polymer microsphere comprising: (i) ibrutinib; and (ii) a biodegradable polymer, wherein each polymer microsphere comprises a drug load of the ibrutinib of greater than 30% by weight of the polymer microsphere, and wherein the polymer microspheres have a particle size of less than 110 μm (D₅₀). In another aspect, the microsphere formulations are characterized in that they have a low initial burst release, that is, not more than 50% of the ibrutinib is released within about 4 hours of injection into a subject.

In one aspect, the microsphere formulations may be made by a method, the method comprising: (A) mixing: (i) the biodegradable polymer; (ii) a primary solvent; and (iii) ibrutinib, to form a dispersed phase; (B) mixing: (i) water; and (ii) a surfactant, to form a continuous phase; and (C) combining the dispersed phase with the continuous phase in a homogenizer.

In one aspect, a method for treating cancer, including a B-cell malignancy, is provided. The method may comprise administering by intramuscular or subcutaneous injection to a patient in need thereof a microsphere formulation made according to the methods described herein.

In another aspect, use is disclosed of a microsphere formulation comprising polymer microspheres, each polymer microsphere comprising: (i) ibrutinib; and (ii) a biodegradable polymer, wherein each polymer microsphere comprises a drug load of the ibrutinib of greater than 30% by weight of the polymer microsphere, and wherein the polymer microspheres have a particle size of less than 110 μm (D₅₀), in the manufacture of a medicament for the treatment of cancer, including a B-cell malignancy.

In another aspect, a microsphere formulation comprising polymer microspheres, each polymer microsphere comprising: (i) ibrutinib; and (ii) a biodegradable polymer, wherein each polymer microsphere comprises a drug load of the ibrutinib of greater than 30% by weight of the polymer microsphere, and wherein the polymer microspheres have a particle size of less than 110 μm (D₅₀), is provided for use as a medicament for the treatment of cancer, including a B-cell malignancy.

In another aspect, a kit is provided, the kit comprising polymer microspheres, each polymer microsphere comprising: (i) ibrutinib; and (ii) a biodegradable polymer, wherein each polymer microsphere comprises a drug load of the ibrutinib of greater than 30% by weight of the polymer microsphere, and wherein the polymer microspheres have a particle size of less than 110 μm (D₅₀).

### BRIEF DESCRIPTION OF THE FIGURES

FIG. 1 is a schematic depicting a method for making ibrutinib-encapsulated polymer microspheres.

FIG. 2 is a graph showing in vitro cumulative ibrutinib release over time from ibrutinib-encapsulating polymer microspheres comprising a 50:50 poly (D,L-lactide-co-glycolide) (“PLGA”) as the biodegradable polymer.

FIG. 3 is a graph showing in vitro cumulative ibrutinib release over time from ibrutinib-encapsulating polymer microspheres comprising a 75:25 PLGA with an inherent viscosity (“IV”) of 0.26 dL/g as the biodegradable polymer.

FIG. 4 is a graph showing in vitro cumulative ibrutinib release over time from ibrutinib-encapsulating polymer microspheres comprising a 75:25 PLGA with IVs between 0.41 dL/g and 0.70 dL/g as the biodegradable polymer.

FIG. 5 is a graph showing in vitro cumulative ibrutinib release over time from ibrutinib-encapsulating polymer microspheres comprising an 85:15 PLGA as the biodegradable polymer.

FIG. 6 is a graph showing in vitro cumulative ibrutinib release over time from ibrutinib-encapsulating polymer microspheres comprising a poly(D,L-lactide) (“PLA”) as the biodegradable polymer.

FIG. 7 is a graph showing in vivo release profiles of several ibrutinib-encapsulating polymer microspheres.

FIG. 8 is a graph showing in vitro cumulative ibrutinib release over time from scaled up Group A and Group A-like release formulations.

FIG. 9 is a graph showing in vitro cumulative ibrutinib release over time from scaled up 28-day release formulations.

### DETAILED DESCRIPTION

Microsphere formulations comprising ibrutinib are provided. The microsphere formulations comprise polymer microspheres, each polymer microsphere comprising: (i) ibrutinib; and (ii) a biodegradable polymer, wherein each polymer microsphere comprises a drug load of the ibrutinib of greater than 30% by weight of the polymer microsphere, and wherein the polymer microspheres have a particle size of less than 110 μm (D₅₀). In another aspect, the microsphere formulations are characterized in that they have a low initial burst release, that is, not more than 20% of the ibrutinib is released within about 24 hours of injection into a subject.

In one aspect, the microsphere formulations may be made by a method, the method comprising: (A) mixing: (i) the biodegradable polymer; (ii) a primary solvent; and (iii) ibrutinib, to form a dispersed phase; (B) mixing: (i) water; and (ii) a surfactant, to form a continuous phase; and (C) combining the dispersed phase with the continuous phase in a homogenizer.

### BTK Inhibitors

In one aspect, the BTK inhibitor is selected from the group comprising, consisting essentially of, or consisting of ibrutinib, acalabrutinib, zanubrutinib, evobrutinib, ABBV-105, fenebrutinib, GS-4059, or spebrutinib, or combinations thereof.

In one aspect, the composition comprises an active pharmaceutical ingredient consisting essentially of ibrutinib. In this context, “consisting essentially of ibrutinib” means that the composition does not contain a sufficient amount (which includes having a zero amount) of a second active pharmaceutical ingredient to have a measurable physiological effect on a condition known to be treatable by administration of such second active pharmaceutical ingredient. In one aspect, the ibrutinib is supplied by ScinoPharm or MSN. In one aspect, the ibrutinib is hydrophobic. In one aspect, the ibrutinib is supplied as a free base. In another aspect, the ibrutinib is supplied as a pharmaceutically acceptable salt. In one aspect, the ibrutinib is characterized by an aqueous solubility of <2.5 mg/g. In one aspect, the ibrutinib is characterized by a solubility in dichloromethane (“DCM”) of >300 mg/g. In one aspect, the ibrutinib is characterized by a pKa of about 3.74.

The ibrutinib may be amorphous, either as a starting material, an intermediate, or in the final polymer microsphere product. The ibrutinib may be crystalline or partially crystalline. The ibrutinib may be crystalline and may exist in the polymer microspheres in various polymorphic forms. Polymorphic forms may include the anhydride, hemihydrates, monohydrates, dihydrates, and other polymorphic forms as known in the art. Salts may include hydrochloride, sulfate, acetate, phosphate, diphosphate, chloride, maleate, citrate, mesylate, nitrate, tartrate, gluconate, or other salts as known in the art.

In an aspect wherein the BTK inhibitor comprises ibrutinib or another BTK inhibitor with similar solubility characteristics, a complex salt may be used to decrease solubility, such as, for example, palmitate, benzoic acid, tosylic acid, camphor-sulfonic acid, or other salt complexes as one of skill in the art can readily envision.

### Biodegradable Polymers

In one aspect, the dispersed phase may include a biodegradable polymer, such as a PLGA or a PLA, although it is contemplated that other suitable biodegradable polymers may be used. The biodegradable polymer may be hydrophobic or hydrophilic.

In some aspects, the biodegradable polymer comprises a PLGA. In one aspect, the PLGA comprises a lactide:glycolide ratio of 50:50, 55:45, 75:25, or 85:15. In one aspect, the PLGA may comprise a blend of PLGAs having different co-monomer ratios, such as, for example, a PLGA having a lactide:glycolide ratio of 55:45 with a PLGA having a lactide:glycolide ratio of 75:25. Of course, any combination of PLGAs having the listed lactide:glycolide ratios (and any ratios therebetween) is contemplated.

In one aspect, the PLGA is acid-terminated. In one aspect, the PLGA is ester-terminated. In one aspect, the PLGA has an IV of from about 0.1 dL/g to about 0.8 dL/g, including from about 0.1 dL/g to about 0.3 dL/g, from about 0.16 dL/g to about 0.24 dL/g, from about 0.2 dL/g to about 0.4 dL/g, from about 0.4 dL/g to about 0.6 dL/g, from about 0.6 dL/g to about 0.8 dL/g, about 0.20 dL/g, 0.26 dL/g, 0.41 dL/g, 0.56 dL/g, 0.66 dL/g, 0.7 dL/g, and any value or range between any two of those IV values.

In one aspect, the PLGA comprises Resomer® 502 H, poly(D,L-lactide-co-glycolide), acid terminated, lactide:glycolide 50:50, manufactured by Evonik, having IV=0.20 (“502 H”). In one aspect, the PLGA comprises Resomer® 502, poly(D,L-lactide-co-glycolide), ester terminated, lactide:glycolide 50:50, manufactured by Evonik, having IV=0.20 (“502”). In one aspect, the PLGA comprises Viatel™ DLG 5503 E, poly (D, L-lactide-co-glycolide), ester terminated, lactide:glycolide 55:45, manufactured by Ashland, having IV=0.20 (“5503 E”). In one aspect, the PLGA comprises Viatel™ DLG 7503 A, poly(D,L-lactide-co-glycolide), acid terminated, lactide:glycolide 75:25, manufactured by Ashland, having IV=0.26 (“7503 A”). In one aspect, the PLGA comprises Viatel™ DLG 7503 E, poly(D,L-lactide-co-glycolide), ester terminated, lactide:glycolide 75:25, manufactured by Ashland, having IV=0.26 (“7503 E”). In one aspect, the PLGA comprises Viatel™ DLG 7505 A, poly(D,L-lactide-co-glycolide), acid terminated, lactide:glycolide 75:25, manufactured by Ashland, having IV=0.56 (“7505 A”). In one aspect, the PLGA comprises Viatel™ DLG 7505 E, poly(D,L-lactide-co-glycolide), ester terminated, lactide:glycolide 75:25, manufactured by Ashland, having IV=0.41 (“7505 E”). In one aspect, the PLGA comprises Viatel™ DLG 7507 A, poly(D,L-lactide-co-glycolide), acid terminated, lactide:glycolide 75:25, manufactured by Ashland, having IV=0.70 (“7507 A”). In one aspect, the PLGA comprises Viatel™ DLG 7507 E, poly(D,L-lactide-co-glycolide), ester terminated, lactide:glycolide 75:25, manufactured by Ashland, having IV=0.66 (“7507 E”). In one aspect, the PLGA comprises Viatel™ DL 8503 A, poly(D,L-lactide-co-glycolide), acid terminated, lactide:glycolide 85:15, manufactured by Ashland, having IV=0.24 (“8503 A”). In one aspect, the PLGA comprises Viatel™ DL 8503 E, poly(D,L-lactide-co-glycolide), ester terminated, lactide:glycolide 85:15, manufactured by Ashland, having IV=0.25 (“8503 E”).

In some aspects, the biodegradable polymer is a PLA. In one aspect, the PLA is acid-terminated. In one aspect, the PLA is ester-terminated. In one aspect, the PLA has an IV of between about 0.1 dL/g and about 0.4 dL/g, including about 0.16 dL/g, about 0.18 dL/g, and about 0.32 dL/g, and any value or range between any two of those IV values.

In one aspect, the PLA comprises a Viatel™ DL 02 A, poly(D,L-lactide), acid terminated, manufactured by Ashland, having IV=0.16 (“DL 02 A”). In one aspect, the PLA comprises a Viatel™ DL 02 E, poly(L-lactide), ester terminated, manufactured by Ashland, having IV=0.18 (“DL 02 E”). In one aspect, the PLA comprises a Viatel™ DL 03 A, poly(L-lactide), acid terminated, manufactured by Ashland, having IV=0.32 (“DL 03 A”).

In one aspect, the biodegradable polymer is mixed with the ibrutinib to form microspheres, which are injectable and formulated to release the ibrutinib to the patient over the intended duration of release. In another aspect, the biodegradable polymer is used to encapsulate the ibrutinib into microspheres, which are injectable and formulated to release the ibrutinib to the patient over the intended duration of release, via a controlled rate of release from the microspheres, or release from different microspheres at different times based upon particle size, thickness of the biodegradable polymer encapsulating the ibrutinib, molecular weight of the biodegradable polymer, polymer composition such as co-monomer ratio, end-cap, and drug load, or combinations of such release-affecting factors.

### Dispersed Phase

In one aspect, the dispersed phase comprises a primary solvent. In one aspect, the primary solvent comprises DCM. The dispersed phase may also include up to about 50% by weight of a co-solvent capable of optimizing the solubility of the ibrutinib in the primary solvent. In one aspect, the primary solvent or the co-solvent may be benzyl alcohol, dimethyl sulfoxide, dimethyl formamide, dimethyl acetamide, acetonitrile, ethanol, N-methyl pyrrolidone, ethyl acetate, tetrahydrofuran, or any other solvent that optimizes the solubility of the ibrutinib in the dispersed phase. A microsphere is “essentially free” of organic solvent if the microsphere meets the standards set forth in the “ICH Harmonised Guideline, Impurities: Guideline for Residual Solvents Q3C(R8), Current Step 4 version dated 22 Apr. 2021,” which is incorporated herein by reference in its entirety.

In some aspects, the dispersed phase may comprise 30% solids (e.g., 15% biodegradable polymer and 15% ibrutinib) or more. In other aspects, the dispersed phase may comprise a lower polymer concentration (e.g., 10% polymer, 10% API).

In some aspects, the dispersed phase may include porogens, such as salt (e.g., NaCl), higher molecular weight solvents, such as polyethylene glycol (Polyethylene Glycol 8000), isopropyl myristate (IPM), and polycaprolactone (PCL), and low molecular weight solvents, such as toluene, hexane, cyclohexanone, 2-ethylhexanol, p-xylene, and n-heptane.

### Continuous Phase

The dispersed phase may be combined with an aqueous continuous phase that comprises water and, optionally, a buffer, a surfactant, or both.

In one aspect, the buffer may be added to the continuous phase to maintain a pH of the solution of about 7.0 to about 8.0. In one aspect, the buffer may be a phosphate buffer or a carbonate buffer. In one aspect, the buffer may be a 10 mM phosphate or carbonate buffer solution and may be used to create and maintain a system pH level of about 7.6.

The surfactant component may be present in the continuous phase in an amount of about 0.35% to about 1.0% by weight in water. In one aspect, the surfactant component comprises polyvinyl alcohol (“PVA”) in a concentration of 0.35% by weight in water.

In some aspects, the dispersed phase flow rate to the homogenizer may be from about 10 mL/min to about 30 mL/min, including about 20 mL/min and about 25 mL/min. In some aspects, the continuous phase flow rate to the homogenizer may be about 2 L/min. Thus, in one aspect, the continuous phase:dispersed phase ratio may be from about 2:1 to about 200:1, including about 40:1, about 66:1, about 80:1, about 100:1, and about 120:1. Larger scale batches may require higher flow rates.

The continuous phase may be provided at room temperature or above or below room temperature. In some aspects, the continuous phase may be provided at about 40° C., about 37° C., about 35° C., about 30° C., about 25° C., about 20° C., about 15° C., about 10° C., about 5° C., about 0° C., and any value or range between any two of those temperature values.

### Homogenizer

For brevity, and because the methods are equally applicable to either, the phrase “homogenizer” contemplates a system or apparatus that can homogenize the dispersed phase and the continuous phase, emulsify the dispersed phase and the continuous phase, or both, which systems and apparatuses are known in the art. For example, in one aspect, the homogenizer is an in-line Silverson Homogenizer (commercially available from Silverson Machines, Waterside UK) or a Levitronix® BPS-i100 integrated pump system used, e.g., as described in U.S. Pat. No. 11,167,256, which is incorporated by reference herein in its entirety. In one aspect, the homogenizer is a membrane emulsifier or a static mixer. In one aspect, the homogenizer runs at an impeller speed of about 1,000 to about 4,000 revolutions per minute (“RPM”), including about 2,000 RPM, about 3,000 RPM, and any value or range between any two of those RPM values.

### Drug Load

The drug load of each polymer microsphere in a drug to polymer ratio, expressed as a percentage, may be greater than 30 wt/wt %, greater than 40 wt/wt %, greater than 50 wt/wt %, greater than 60 wt/wt %, or greater than 70 wt/wt %, including from about 30 wt/wt % to about 75 wt/wt %, from about 35 wt/wt % to about 70 wt/wt %, from about 40 wt/wt % to about 65 wt/wt %, from about 45 wt/wt % to about 60 wt/wt %, about 30 wt/wt %, about 35 wt/wt %, about 40 wt/wt %, about 45 wt/wt %, about 50 wt/wt %, about 60 wt/wt %, about 65 wt/wt %, about 70 wt/wt %, about 75 wt/wt %, and any value or range between any two of those drug loads.

In some particular aspects, it is contemplated that the drug load may be as low as 20 wt/wt %.

### Particle Size

In one aspect, the polymer microspheres may have a particle size of less than 110 μm (D₅₀), including between about 20 μm (D₅₀) and about 60 μm (D₅₀), between about 30 μm (D₅₀) and about 50 μm (D₅₀), between about 30 μm (D₅₀) and about 40 μm (D₅₀), between about 35 μm (D₅₀) and about 60 μm (D₅₀), between about 45 μm (D₅₀) and about 60 μm (D₅₀), about 20 μm (D₅₀), about 25 μm (D₅₀), about 30 μm (D₅₀), about 35 μm (D₅₀), about 40 μm (D₅₀), about 45 μm (D₅₀), about 50 μm (D₅₀), about 55 μm (D₅₀), about 60 μm (D₅₀), less than about 60 μm (D₅₀), and any value or range between any two of those particle sizes.

In some particular aspects, it is contemplated that particle sizes may be as large as 150-200 μm.

### Extended Release

In one aspect, the microsphere formulations are characterized in that they have an in vivo duration of release of less than about 7 days in humans. In one aspect, the microsphere formulations are characterized in that they have an in vivo duration of release of between about 7 days to about 14 days in humans. In one aspect, the microsphere formulations are characterized in that they have an in vivo duration of release of between about 14 days to about 28 days in humans. In one aspect, the microsphere formulations are characterized in that they have an in vivo duration of release of about 28 days in humans. In one aspect, the microsphere formulations are characterized in that they have an in vivo duration of release of greater than about 28 days in humans.

In one aspect, the microsphere formulations are characterized in that at least about 50%, at least about 60%, at least about 70%, at least about 80%, at least about 90%, or 100%, and any range between any of those values, of the ibrutinib is released within <7, 7-14, 14-28, or >28 days (as described in the preceding paragraph) of injection into a subject. For example, in one aspect, the microsphere formulations are characterized in that about 75% to 100% of the ibrutinib is released over the designated period after injection into a subject. In another aspect, the microsphere formulations are characterized in that they have a low initial burst release, that is, not more than about 20% of the ibrutinib is released within about 24 hours of injection into a subject.

In another aspect, mixed release profile microsphere formulations comprising ibrutinib are provided. In one aspect, the mixed release profile microsphere formulations comprise: (i) first polymer microspheres that are characterized by a release of ibrutinib above a therapeutic level for an initial period; (ii) second polymer microspheres that are characterized by a release of ibrutinib above a therapeutic level for an intermediate period at or near the end of the initial period, but which may overlap with the initial period; and, optionally, (iii) third polymer microspheres that are characterized by a release of ibrutinib above a therapeutic level for a final period at or near the end of the intermediate period, but which may overlap with the intermediate period. An example of a mixed release profile microsphere formulation can be seen in U.S. Provisional Patent Application No. 63/381,696, the entire disclosure of which is incorporated herein by reference.

A further aspect includes a sustained release injectable formulation of ibrutinib that is pharmacologically comparable to oral doses of: 70 mg, 140 mg, 280 mg, 420 mg, and 560 mg, in sustained release injectable formulations that release over approximately 7, 14, or 28 days.

Another aspect includes a method of treating a human patient for MCL, CLL/SLL, and other diseases or conditions that may be treated by the ibrutinib. The method may comprise providing an injectable form of ibrutinib in a dosage strength that is pharmacologically comparable to 70 mg, 140 mg, 280 mg, 420 mg, and 560 mg per day orally, the injectable form with a duration of continuous release such that patient compliance is assured, the medical consequences of missing a dose or doses are avoided, and the pharmacokinetic profile is improved as compared with the oral dosage form.

### Therapeutic Benefits

Possible conditions that may be treated using the microsphere formulations comprising ibrutinib include cancer, including B-cell malignancies, including MCL, CCL, and SLL. In one aspect, a B-cell malignancy may be treated using the microsphere formulations comprising ibrutinib, wherein the microsphere formulations are administered about every <7, 7-14, 14-28, or >28 days.

In one aspect, a method for treating cancer, including a B-cell malignancy, is provided. The method may comprise administering by intramuscular or subcutaneous injection to a patient in need thereof a microsphere formulation made according to the methods described herein.

In another aspect, use is disclosed of a microsphere formulation comprising polymer microspheres, each polymer microsphere comprising: (i) ibrutinib; and (ii) a biodegradable polymer, wherein each polymer microsphere comprises a drug load of the ibrutinib of greater than 30% by weight of the polymer microsphere, and wherein the polymer microspheres have a particle size of less than 110 μm (D₅₀), in the manufacture of a medicament for the treatment of cancer, including a B-cell malignancy.

In another aspect, a microsphere formulation comprising polymer microspheres, each polymer microsphere comprising: (i) ibrutinib; and (ii) a biodegradable polymer, wherein each polymer microsphere comprises a drug load of the ibrutinib of greater than 30% by weight of the polymer microsphere, and wherein the polymer microspheres have a particle size of less than 110 μm (D₅₀), is provided for use as a medicament for the treatment of cancer, including a B-cell malignancy.

In another aspect, a kit is provided, the kit comprising polymer microspheres, each polymer microsphere comprising: (i) ibrutinib; and (ii) a biodegradable polymer, wherein each polymer microsphere comprises a drug load of the ibrutinib of greater than 30% by weight of the polymer microsphere, and wherein the polymer microspheres have a particle size of less than 110 μm (D₅₀).

### EXAMPLES

### Example 1—General Preparation of Polymer Microspheres Comprising Ibrutinib

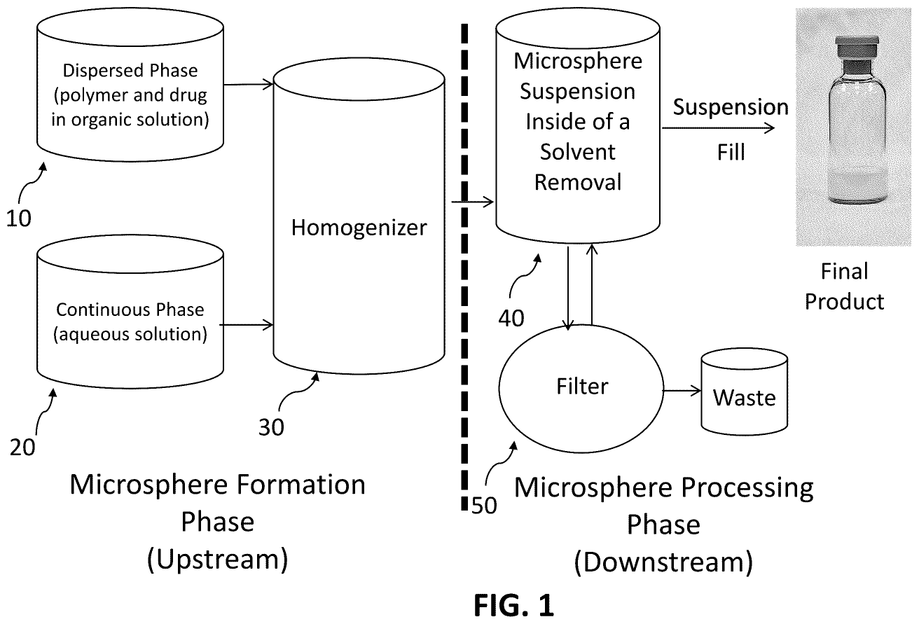

Microsphere Formation Phase. With reference to FIG. 1, a dispersed phase (“DP”) 10 is formed by dissolving a polymer matrix (such as a PLGA or PLA polymer) in an organic solvent system (such as DCM), followed by the addition of ibrutinib with mixing until completely dissolved. The DP 10 is filtered using a 0.2 μm sterilizing PTFE or PVDF membrane filter (such as EMFLON, commercially available from Pall or SartoriousAG) and pumped into a homogenizer 30 at a defined flow rate. A continuous phase (“CP”) 20 comprising water, surfactant, and, optionally, a buffer is also pumped into the homogenizer 30 at a defined flow rate. The speed of the homogenizer 30 is generally fixed to achieve a desired polymer microsphere size distribution. A representative continuous “upstream” microsphere formation phase is described in U.S. Pat. No. 5,945,126, which is incorporated by reference herein in its entirety.

Microsphere Processing Phase. The formed or forming microspheres exit the homogenizer 30 and enter a solvent removal vessel (“SRV”) 40. Water may be added to the SRV 40 during microsphere formation to minimize the solvent level in the aqueous medium. See, e.g., U.S. Pat. No. 9,017,715, which is incorporated by reference herein in its entirety. After the DP 10 has been exhausted, the CP 20 and water flow rates are stopped, and the washing steps are initiated. Solvent removal is achieved using water washing and a hollow fiber filter (commercially available as HFF from Cytiva) 50. A representative “downstream” microsphere processing phase is described in U.S. Pat. No. 6,270,802, which is incorporated by reference herein in its entirety.

The washed microspheres are collected and freeze-dried in a lyophilizer (Virtis) to remove any moisture. The resulting microspheres are a free-flowing off-white bulk powder.

A double emulsion method is also contemplated. The method may comprise: (i) contacting ibrutinib with a biodegradable PLGA polymer in the presence of a solvent to form an organic component and providing the organic component to a first homogenizer; (ii) providing an inner aqueous component comprising water and optionally a first surfactant to the first homogenizer; (iii) homogenizing the organic component with the inner aqueous component to form a primary emulsion; (iv) providing the primary emulsion to a second homogenizer at a first flow rate; (v) providing a continuous phase comprising water and optionally a second surfactant to the second homogenizer at a second flow rate; (vi) homogenizing the primary emulsion and the continuous phase; and (iv) removing the solvent to form the polymer microspheres, wherein each of the formed polymer microspheres incorporates at least a portion of the inner aqueous component in the form of a plurality of emulsions. An example of a double emulsion method may be seen in U.S. Patent Application Publication No. US20220054420A1, the entire disclosure of which is incorporated by reference herein.

### Example 2—Preparation of Ibrutinib-Encapsulated Polymer Microspheres Comprising a 50:50 PLGA—Batch Nos. 1, 2, and 28 (“Group A”)

Following the general procedure described in Example 1 and illustrated in FIG. 1, the DP was formed by dissolving 2.5 g of either 502 H polymer (Batch No. 1) or 502 polymer (Batch Nos. 2 and 28) in 11.67 g of DCM, followed by addition of ibrutinib (2.5 g) with mixing until completely dissolved. The DP was filtered and pumped at a flow rate of 25 mL/min into a Levitronix® BPS-i100 integrated pump system operating at 3,000 RPM. The CP comprising 0.35% PVA was also pumped into the homogenizer at a flow rate of 2 L/min (CP:DP=80:1).

The formed or forming microspheres exited the homogenizer and entered the SRV. Deionized water was added to the SRV. Solvent removal was achieved using water washing and a hollow fiber filter. The bulk suspension was collected via filtration and lyophilized to obtain a free-flowing powder.

Batch No. 1 had a particle size of 36 μm (D₅₀), a drug load of 47.6 wt %, and a molecular weight of 17.6 kDa. The microspheres contained residual DCM of 3.0%. Batch No. 2 had a particle size of 44 μm (D₅₀), a drug load of 47.8 wt %, and a molecular weight of 17.7 kDa. The microspheres contained residual DCM of 3.0%. Batch No. 28 had a particle size of 33 μm (D₅₀), a drug load of 49.4 wt %, and a molecular weight of 15.6 kDa. The microspheres contained residual DCM of 0.2%. Batch 2 and Batch 28 differ in their washing protocol, with Batch No. 2 subject to a room temperature wash for 60 min, and Batch No. 28 subject to a room temperature wash for 20 min, followed by a 40 min wash at 35-39° C. The parameters and results are shown tabularly in Table 1:

TABLE 1

| Lot | Batch Number | | 1 | 2 | 28 |
|---|---|---|---|---|---|
| | Symbol | | ∘ | Δ | ▭ |
| Formulations | Polymer Supplier/Name | | Evonik | | |
| | Co-Monomer Ratio | | 50:50 | | |
| | Polymer IV (dL/g) | | 0.20 | | |
| | Polymer Endcap | | Acid | Ester | |
| | Batch Size (g) | | 5 | | |
| | Mixing Speed (RPM) | | 3000 | | |
| | Target Drug Load (%) | | 50.0 | | |
| Analytical | Drug Load (%) | | 47.6 | 47.8 | 49.4 |
| | Encapsulation Efficiency (%) | | 95.2 | 95.6 | 98.8 |
| | Residual Solvent DCM (%) | | 3.0 | 3.0 | 0.2 |
| | Particle | Dᵥ10 | 17 | 18 | 12 |
| | Size | Dᵥ50 | 36 | 44 | 33 |
| | (μm) | Dᵥ90 | 59 | 79 | 57 |

| | | | |
|---|---|---|---|
| Sample MW (kDa) | 17.6 | 17.7 | 15.6 |
| Polymer MW (kDa) | 17.4 | 17.6 | 15.7 |

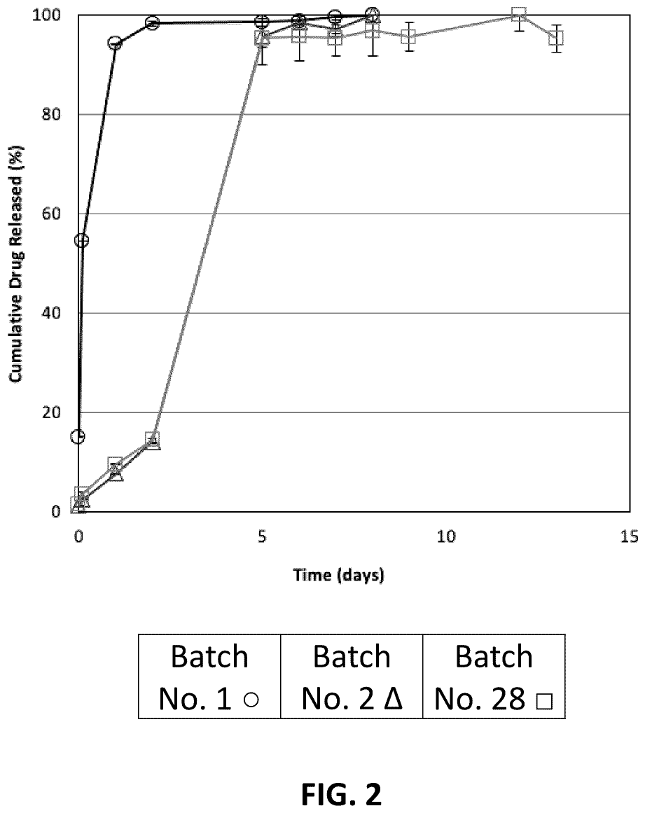

FIG. 2 is a graph showing in vitro cumulative ibrutinib release over time from Group A ibrutinib-encapsulating polymer microspheres.

### Example 3—Preparation of Ibrutinib-Encapsulated Polymer Microspheres Comprising a 75:25 PLGA with a Low Polymer IV—Batch Nos. 3, 4, 6, 7, and 11 (“Group B”)

Following the general procedure described in Example 1 and illustrated in FIG. 1, the DP was formed by dissolving 2.5 g (Batch Nos. 3, 4, and 11), 2.0 g (Batch No. 6), or 1.5 g (Batch No. 7) of either 7503 A polymer (Batch Nos. 3, 6, 7, and 11) or 7502 E polymer (Batch No. 4) (IV=0.26 dL/g) in 11.67 g of DCM, followed by addition of ibrutinib (sufficient to provide a DP weight of 16.67 g, i.e., 2.5 g for Batch Nos. 3, 4, and 11; 3.0 g for Batch No. 6; and 3.5 g for Batch No. 7) with mixing until completely dissolved. The DP was filtered and pumped at a flow rate of 25 mL/min into a Levitronix® BPS-i100 integrated pump system operating at 3,000 RPM (Batch Nos. 3, 4, 6, and 7) or 2,000 RPM (Batch No. 11). The CP comprising 0.35% PVA was also pumped into the homogenizer at a flow rate of 2 L/min (CP:DP=80:1).

The formed or forming microspheres exited the homogenizer and entered the SRV. Deionized water was added to the SRV. Solvent removal was achieved using water washing and a hollow fiber filter. The bulk suspension was collected via filtration and lyophilized to obtain a free-flowing powder.

Batch No. 3 had a particle size of 39 μm (D₅₀), a drug load of 48.2 wt %, and a molecular weight of 29.4 kDa. The microspheres contained residual DCM of 3.1%. Batch No. 4 had a particle size of 35 μm (D₅₀), a drug load of 48.9 wt %, and a molecular weight of 25.5 kDa. The microspheres contained residual DCM of 2.1%. Batch No. 6 had a particle size of 34 μm (D₅₀), a drug load of 60.6 wt %, and a molecular weight of 31.0 kDa. The microspheres contained residual DCM of 2.7%. Batch No. 7 had a particle size of 30 μm (D₅₀), a drug load of 65.6 wt %, and a molecular weight of 30.1 kDa. The microspheres contained residual DCM of 1.5%. Batch No. 11 had a particle size of 61 μm (D₅₀), a drug load of 51 wt %, and a molecular weight of 28.9 kDa. The microspheres contained residual DCM of 1.4%. The parameters and results are shown tabularly in Table 2:

TABLE 2

| | Batch Number | | 3 | 4 | 6 | 7 | 11 |
|---|---|---|---|---|---|---|---|
| Lot | Symbol | | ◯ | Δ | □ | ⋄ | X |
| Formulations | Polymer | | Ashland | | | | |
| | Supplier/Name | | | | | | |
| | Co-Monomer | | 75:25 | | | | |
| | Ratio | | | | | | |
| | Polymer IV | | 0.26 | | | | |
| | (dL/g) | | | | | | |
| | Polymer | | Acid | Ester | Acid | | |
| | Endcap | | | | | | |
| | Batch Size (g) | | 5 | | | | |
| | Mixing Speed | | 3000 | | | | 2000 |
| | (RPM) | | | | | | |
| | Target Drug | | 50.0 | | 60.0 | 70.0 | 50.0 |
| | Load (%) | | | | | | |
| Analytical | Drug Load | | 48.2 | 48.9 | 60.6 | 65.6 | 51.0 |
| | (%) | | | | | | |
| | Encapsulation | | 96.4 | 97.8 | 101.0 | 93.7 | 102.0 |
| | Efficiency (%) | | | | | | |
| | Residual | | 3.1 | 2.1 | 2.7 | 1.5 | 1.4 |
| | Solvent DCM | | | | | | |
| | (%) | | | | | | |
| | Particle | Dᵥ10 | 17 | 15 | 12 | 10 | 29 |
| | Size | Dᵥ50 | 39 | 35 | 34 | 30 | 61 |
| | (μm) | Dᵥ90 | 65 | 59 | 58 | 53 | 103 |

| | | | | | |
|---|---|---|---|---|---|
| Sample MW | 29.4 | 25.5 | 31.0 | 30.1 | 28.9 |
| (kDa) | | | | | |
| Polymer MW | 30.7 | 25.8 | 30.7 | 30.7 | 28.6 |
| (kDa) | | | | | |

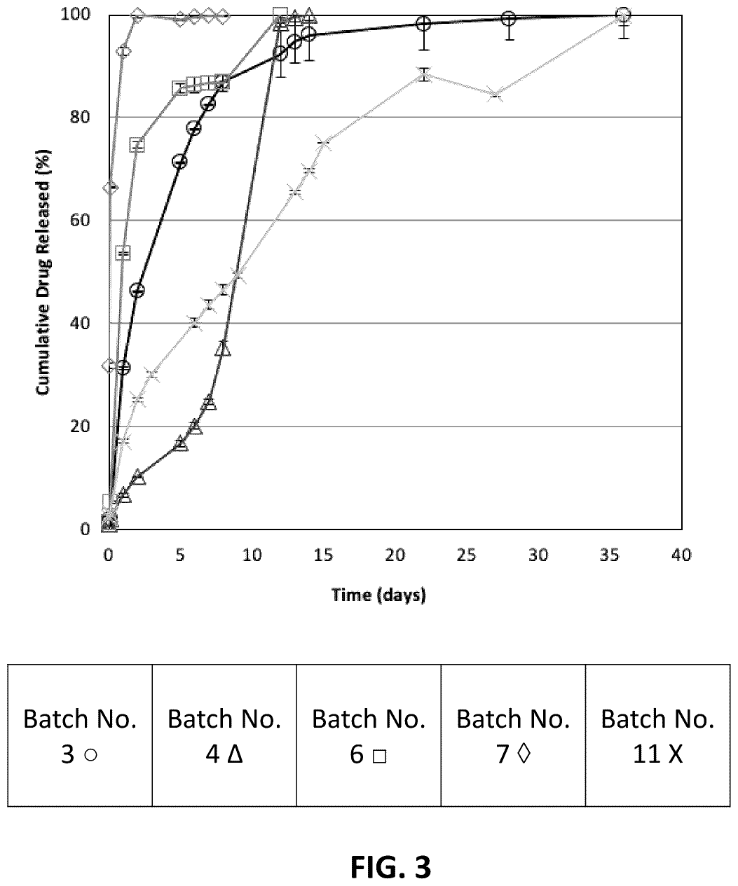

FIG. 3 is a graph showing in vitro cumulative ibrutinib release over time from Group B (Batch Nos. 3, 4, 6, 7, and 11) ibrutinib-encapsulating polymer microspheres.

### Example 4—Preparation of Ibrutinib-Encapsulated Polymer Microspheres Comprising a 75:25 PLGA with a High Polymer IV—Batch Nos. 5, 12, 13, 14, and 30 (“Group C”)

Following the general procedure described in Example 1 and illustrated in FIG. 1, the DP was formed by dissolving 2.5 g (Batch Nos. 5, 12, and 30) or 2.0 g (Batch Nos. 13 and 14) of either 7505 A polymer (Batch Nos. 5 and 30) (IV=0.56 dL/g), 7505 E polymer (Batch No. 12) (IV=0.41 dL/g), 7507 A polymer (Batch No. 13) (IV=0.7 dL/g), or 7507 E polymer (Batch No. 14) (IV=0.66 dL/g) in 11.67 g of DCM, followed by addition of ibrutinib (sufficient to provide a DP weight of 16.67 g, i.e., 2.5 g for Batch Nos. 5, 12, and 30; and 3.0 g for Batch Nos. 13 and 14) with mixing until completely dissolved. The DP was filtered and pumped at a flow rate of 25 mL/min into a Levitronix® BPS-i100 integrated pump system operating at 3,000 RPM. The CP comprising 0.35% PVA was also pumped into the homogenizer at a flow rate of 2 L/min (CP:DP=80:1).

The formed or forming microspheres exited the homogenizer and entered the SRV. Deionized water was added to the SRV. Solvent removal was achieved using water washing and a hollow fiber filter. The bulk suspension was collected via filtration and lyophilized to obtain a free-flowing powder.

Batch No. 5 had a particle size of 53 μm (D₅₀), a drug load of 47.5 wt %, and a molecular weight of 66.4 kDa. The microspheres contained residual DCM of 4.1%. Batch No. 12 had a particle size of 47 μm (D₅₀), a drug load of 51.2 wt %, and a molecular weight of 49.8 kDa. The microspheres contained residual DCM of 0.8%. Batch No. 13 had a particle size of 52 μm (D₅₀), a drug load of 62.2 wt %, and a molecular weight of 87.7 kDa. The microspheres contained residual DCM of 1.3%. Batch No. 14 had a particle size of 53 μm (D₅₀), a drug load of 61.0 wt %, and a molecular weight of 90.8 kDa. Batch No. 30 had a particle size of 46 μm (D₅₀), a drug load of 48.8 wt %, and a molecular weight of 46.2 kDa. The microspheres contained residual DCM of 1.2%. The microspheres contained residual DCM of 1.3%. The parameters and results are shown tabularly in Table 3:

TABLE 3

| | Batch Number | | 5 | 12 | 13 | 14 | 30 |
|---|---|---|---|---|---|---|---|
| Lot | Symbol | | ◯ | Δ | □ | ⋄ | + |
| Formulations | Polymer | | Ashland | | | | |
| | Supplier/Name | | | | | | |
| | Co-Monomer | | 75:25 | | | | |
| | Ratio | | | | | | |
| | Polymer IV | | 0.56 | 0.41 | 0.70 | 0.66 | 0.41 |
| | (dL/g) | | | | | | |
| | Polymer | | Acid | Ester | Acid | Ester | Acid |
| | Endcap | | | | | | |
| | Batch Size (g) | | 5 | | | | |
| | Mixing Speed | | 3000 | | | | |
| | (RPM) | | | | | | |
| | Target Drug | | 50.0 | | 60.0 | | 50.0 |
| | Load (%) | | | | | | |
| Analytical | Drug Load | | 47.5 | 51.2 | 62.2 | 61.0 | 49.8 |
| | (%) | | | | | | |
| | Encapsulation | | 95.0 | 102.4 | 103.7 | 101.7 | 99.6 |
| | Efficiency (%) | | | | | | |
| | Residual | | 4.1 | 0.8 | 1.3 | 1.3 | 1.2 |
| | Solvent DCM | | | | | | |
| | (%) | | | | | | |
| | Particle | Dᵥ10 | 15 | 17 | 14 | 15 | 12 |
| | Size | Dᵥ50 | 53 | 47 | 52 | 53 | 46 |
| | (μm) | Dᵥ90 | 102 | 84 | 106 | 106 | 89 |

| | | | | | |
|---|---|---|---|---|---|
| Sample MW | 66.4 | 49.8 | 87.7 | 90.8 | 46.2 |
| (kDa) | | | | | |
| Polymer MW | 66.2 | 50.0 | 91.0 | 92.9 | 45.8 |
| (kDa) | | | | | |

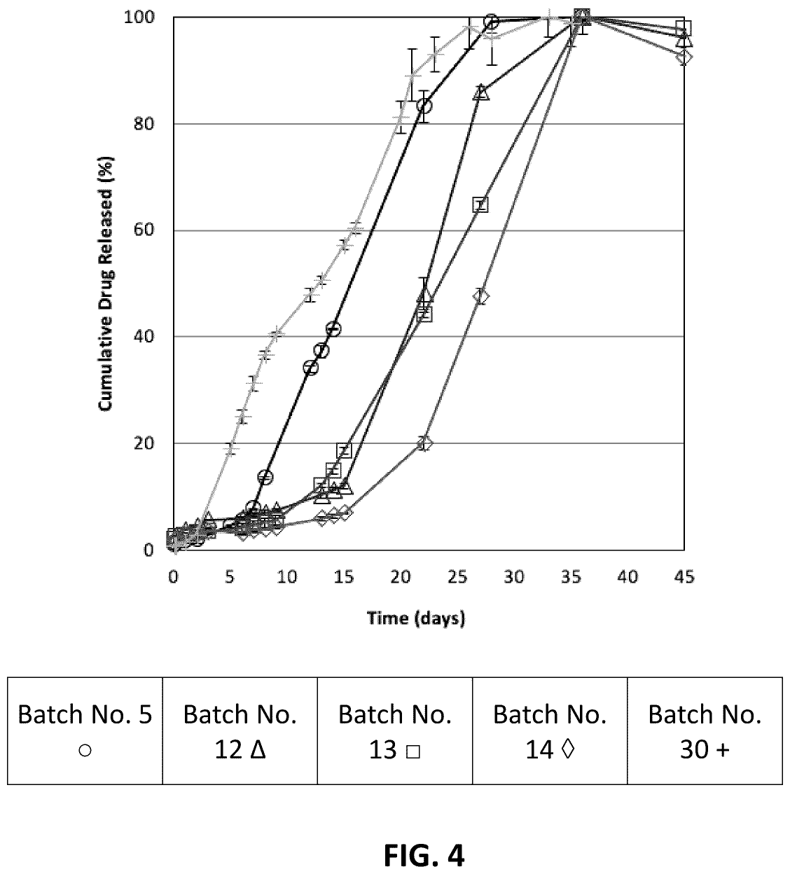

FIG. 4 is a graph showing in vitro cumulative ibrutinib release over time from the Group C ibrutinib-encapsulating polymer microspheres.

### Example 5—Preparation of Ibrutinib-Encapsulated Polymer Microspheres Comprising an 85:15 PLGA—Batch Nos. 18 and 19 (“Group D”)

Following the general procedure described in Example 1 and illustrated in FIG. 1, the DP was formed by dissolving 2.5 g of either 8503 A polymer (Batch No. 18) (IV=0.24 dL/g) or 8503 E polymer (Batch No. 19) (IV=0.25 dL/g) in 11.67 g of DCM, followed by addition of ibrutinib (2.5 g) with mixing until completely dissolved. The DP was filtered and pumped at a flow rate of 25 mL/min into a Levitronix® BPS-i100 integrated pump system operating at 3,000 RPM. The CP comprising 0.35% PVA was also pumped into the homogenizer at a flow rate of 2 L/min (CP:DP=80:1).

The formed or forming microspheres exited the homogenizer and entered the SRV. Deionized water was added to the SRV. Solvent removal was achieved using water washing and a hollow fiber filter. The bulk suspension was collected via filtration and lyophilized to obtain a free-flowing powder.

Batch No. 18 had a particle size of 36 μm (D₅₀), a drug load of 49.8 wt %, and a molecular weight of 22.7 kDa. The microspheres contained residual DCM of 0.5%. Batch No. 19 had a particle size of 35 μm (D₅₀), a drug load of 49.2 wt %, and a molecular weight of 25.8 kDa. The microspheres contained residual DCM of 0.3%. The parameters and results are shown tabularly in Table 4:

TABLE 4

| Lot | Batch Number | | 18 | 19 |
|---|---|---|---|---|
| | Symbol | | ∘ | Δ |
| Formulations | Polymer Supplier/Name | | Ashland | |
| | Co-Monomer Ratio | | 85:15 | |
| | Polymer IV (dL/g) | | 0.24 | 0.25 |
| | Polymer Endcap | | Acid | Ester |
| | Batch Size (g) | | 5 | |
| | Mixing Speed (RPM) | | 3000 | |
| | Target Drug Load (%) | | 50 | |
| Analytical | Drug Load (%) | | 49.8 | 49.2 |
| | Encapsulation Efficiency (%) | | 99.6 | 98.4 |
| | Residual Solvent DCM (%) | | 0.5 | 0.3 |
| | Particle | Dᵥ10 | 15 | 13 |
| | Size | Dᵥ50 | 36 | 35 |
| | (μm) | Dᵥ90 | 62 | 61 |

| | | |
|---|---|---|
| Sample MW (kDa) | 22.7 | 25.8 |
| Polymer MW (kDa) | 22.7 | 26.4 |

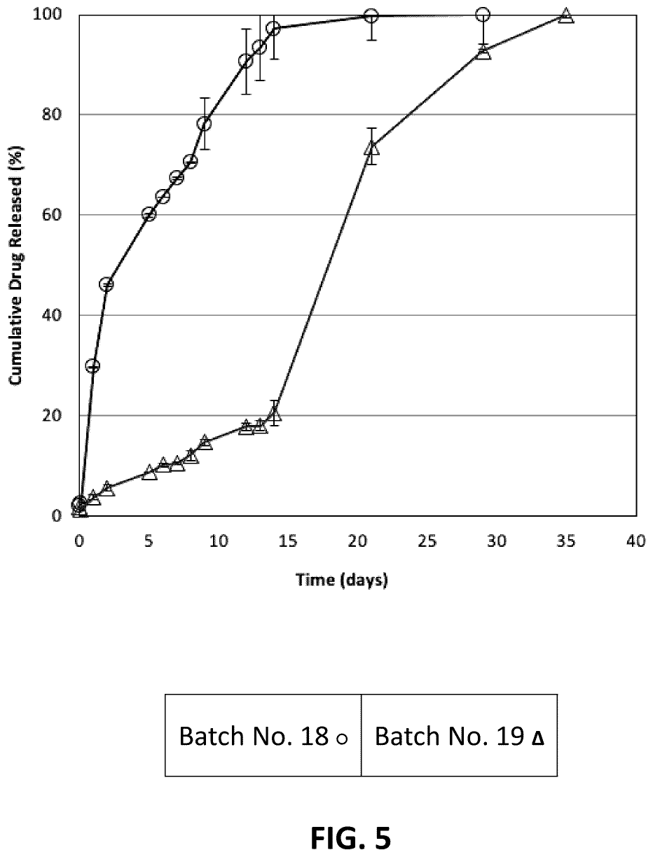

FIG. 5 is a graph showing in vitro cumulative ibrutinib release over time from the Group D ibrutinib-encapsulating polymer microspheres.

### Example 6—Preparation of Ibrutinib-Encapsulated Polymer Microspheres Comprising a PLA—Batch Nos. 8, 9, 10, 16, and 17 (“Group E”)

Following the general procedure described in Example 1 and illustrated in FIG. 1, the DP was formed by dissolving 2.5 g (Batch Nos. 8, 9, 16, and 17) or 2.0 g (Batch No. 10) of either DL 02 A polymer (Batch Nos. 8 and 17) (IV=0.16 dL/g), DL 02 E polymer (Batch Nos. 9 and 10) (IV=0.18 dL/g), or DL 03 A polymer (Batch No. 16) (IV=0.32 dL/g) in 11.67 g of DCM, followed by addition of ibrutinib (sufficient to provide a DP weight of 16.67 g, i.e., 2.5 g for Batch Nos. 8, 9, 16, and 17; and 3.0 g for Batch No. 10) with mixing until completely dissolved. The DP was filtered and pumped at a flow rate of 25 mL/min into a Levitronix® BPS-i100 integrated pump system operating at either 3,000 RPM (Batch Nos. 8, 9, 10, and 16) or 2,000 RPM (Batch No. 17). The CP comprising 0.35% PVA was also pumped into the homogenizer at a flow rate of 2 L/min (CP:DP=80:1).

The formed or forming microspheres exited the homogenizer and entered the SRV. Deionized water was added to the SRV. Solvent removal was achieved using water washing and a hollow fiber filter. The bulk suspension was collected via filtration and lyophilized to obtain a free-flowing powder.

Batch No. 8 had a particle size of 32 μm (D₅₀), a drug load of 51.7 wt %, and a molecular weight of 12.0 kDa. The microspheres contained residual DCM of 0.4%. Batch No. 9 had a particle size of 29 μm (D₅₀), a drug load of 51.8 wt %, and a molecular weight of 11.7 kDa. The microspheres contained residual DCM of 0.1%. Batch No. 10 had a particle size of 29 μm (D₅₀), a drug load of 64.2 wt %, and a molecular weight of 11.7 kDa. The microspheres contained residual DCM of 0.2%. Batch No. 16 had a particle size of 40 μm (D₅₀), a drug load of 48.9 wt %, and a molecular weight of 30.1 kDa. The microspheres contained residual DCM of 0.6%. Batch No. 17 had a particle size of 49 μm (D₅₀), a drug load of 50.2 wt %, and a molecular weight of 12.4 kDa. The microspheres contained residual DCM of 0.8%. The parameters and results are shown tabularly in Table 5:

TABLE 5

| | Batch Number | | 8 | 9 | 10 | 16 | 17 |
|---|---|---|---|---|---|---|---|
| Lot | Symbol | | ◯ | Δ | □ | ⋄ | X |
| Formulations | Polymer | | Ashland | | | | |
| | Supplier/Name | | | | | | |
| | Co-Monomer | | 100:0 | | | | |
| | Ratio | | | | | | |
| | Polymer IV | | 0.16 | 0.18 | | 0.32 | 0.16 |
| | (dL/g) | | | | | | |
| | Polymer | | Acid | Ester | | Acid | |
| | Endcap | | | | | | |
| | Batch Size (g) | | 5 | | | | |
| | Mixing Speed | | 3000 | | | | 2000 |
| | (RPM) | | | | | | |
| | Target Drug | | 50.0 | | 60.0 | 50.0 | |
| | Load (%) | | | | | | |
| Analytical | Drug Load | | 51.7 | 51.8 | 64.2 | 48.9 | 50.2 |
| | (%) | | | | | | |
| | Encapsulation | | 103.4 | 103.6 | 107.0 | 97.8 | 100.4 |
| | Efficiency (%) | | | | | | |
| | Residual | | 0.4 | 0.1 | 0.2 | 0.6 | 0.8 |
| | Solvent DCM | | | | | | |
| | (%) | | | | | | |
| | Particle | Dᵥ10 | 15 | 12 | 14 | 18 | 25 |
| | Size | Dᵥ50 | 32 | 29 | 29 | 40 | 49 |
| | (μm) | Dᵥ90 | 53 | 50 | 50 | 66 | 81 |

| | | | | | |
|---|---|---|---|---|---|
| Sample MW | 12.0 | 11.7 | 11.7 | 30.1 | 12.4 |
| (kDa) | | | | | |
| Polymer MW | 11.9 | 11.8 | 11.8 | 29.5 | 11.7 |
| (kDa) | | | | | |

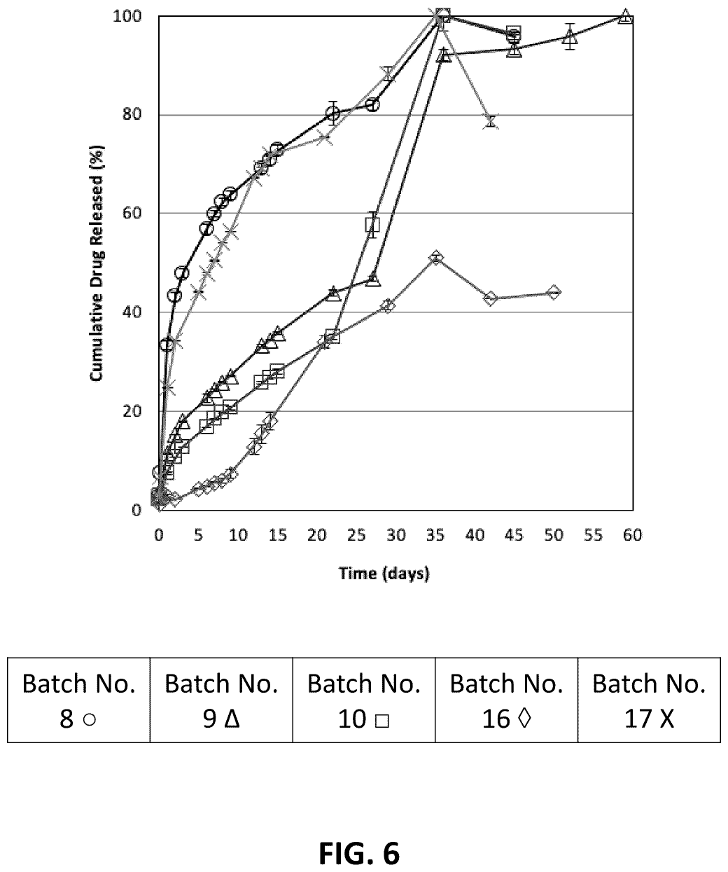

FIG. 6 is a graph showing in vitro cumulative ibrutinib release over time from the Group E ibrutinib-encapsulating polymer microspheres.

### Example 7—Pharacokinetics Study in Rats of Batch Nos. 28, 18, 17, 30, and 14 (the “PK Study Formulations”)

The pharmacokinetic profile of ibrutinib following a subcutaneously injected dose of the PK study formulations in rats was studied. One goal of the study was to determine the in vitro—in vivo correlations using formulations with different polymer co-monomer ratios and that span a range ofrelease rates.

Five male rats per group (25 total rats) received a 30 mg/kg dose (dose volume 1.5 mL/kg) of the stated Batch No. Blood was collected pre-dose, at 0.5, 1, 6, 12, 24, 48, and 96 hours, and at 7, 14, 21, 28, 35, 42, and 49 days (25 rats×14 samples/rat 350 samples).

The parameters of the five PK study formulations are shown together in Table 6:

TABLE 6

| | Batch Number | | 28 | 18 | 17 | 30 | 14 |
|---|---|---|---|---|---|---|---|
| Lot | Symbol | | ◯ | Δ | □ | ⋄ | X |
| Polymer | Polymer | | Evonik | Ashland | | | |
| | Supplier/Name | | | | | | |
| | Co-Monomer | | 50:50 | 85:15 | 100:0 | 75:25 | |
| | Ratio | | | | | | |
| | Polymer IV | | 0.20 | 0.24 | 0.16 | 0.41 | 0.66 |
| | (dL/g) | | | | | | |
| | Polymer | | Ester | Acid | | | Ester |
| | Endcap | | | | | | |
| Form- | Batch Size (g) | | 5 | | | | |
| ulations | Total Yield (%) | | 59 | 67 | 69 | 40 | 36 |
| | Mixing Speed | | 3000 | | | | 2000 |
| | (RPM) | | | | | | |
| | Target Drug | | 50.0 | | | | 60.0 |
| | Load (%) | | | | | | |
| Ana- | Drug Load (%) | | 49.4 | 49.8 | 50.2 | 49.8 | 61.0 |
| lytical | Encapsulation | | 98.8 | 99.6 | 100.4 | 99.6 | 101.7 |
| | Efficiency (%) | | | | | | |
| | Residual | | 0.2 | 0.5 | 0.8 | 1.2 | 1.3 |
| | Solvent | | | | | | |
| | DCM (%) | | | | | | |
| | Particle | Dᵥ10 | 12 | 15 | 25 | 12 | 15 |
| | Size | Dᵥ50 | 33 | 36 | 49 | 46 | 53 |
| | (μm) | Dᵥ90 | 57 | 62 | 81 | 89 | 106 |

| | | | | | |
|---|---|---|---|---|---|
| Sample MW | 15.6 | 22.7 | 12.4 | 46.2 | 90.8 |
| (kDa) | | | | | |
| Polymer MW | 15.7 | 22.7 | 11.7 | 45.8 | 92.9 |
| (kDa) | | | | | |

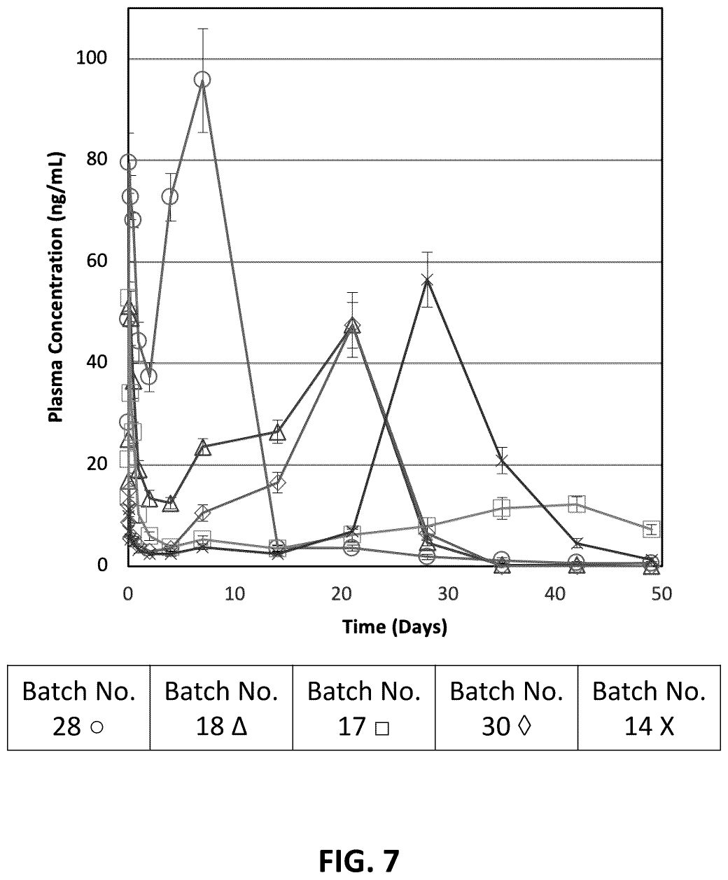

FIG. 7 is a graph showing the in vivo release profiles of the PK study formulations. All batches had around a 2-5× difference in initial peak to “steady state.” Batch No. 28 exhibited the highest burst. PLGA formulations burst twice, first at the time of initial injection and again later, which is a common trend for PLGA degradation. The plasma concentration levels for Batch Nos. 28, 18, and 30 were undetectable by day 35.

### Example 8—Scale Up and Optimization of Group a and Group A-Like Ibrutinib-Encapsulated Polymer Microspheres

Following the general procedure described in Example 1 and illustrated in FIG. 1, for both Batch Nos. 34 and 36, the DP was formed by dissolving 250 g of DLG 5503 E polymer, followed by addition of ibrutinib (250 g) with mixing until completely dissolved. The DP was filtered and pumped at a flow rate of 25 mL/min into a Levitronix® BPS-i100 integrated pump system operating at 3,000 RPM. The CP comprising 0.35% PVA was also pumped into the homogenizer at a flow rate of 2 L/min (CP:DP=80:1).

The formed or forming microspheres exited the homogenizer and entered the SRV. Deionized water was added to the SRV. Solvent removal was achieved using water washing and filtering. The bulk suspension was collected via filtration and lyophilized to obtain a free-flowing powder.

Batch No. 34 had a particle size of 37 μm (D₅₀), a drug load of 50.4 wt %, and a molecular weight of 20.2 kDa. The microspheres contained residual DCM of 0.4%. Batch No. 36, which was further purified and vialed, had a particle size of 37 μm (D₅₀), a drug load of 48.3 wt %, and a molecular weight of 23.2 kDa. The microspheres contained residual DCM of 0.0%. The parameters and results are shown tabularly in Table 7, relative to Batch No. 28:

TABLE 7

| Lot | Batch Number | | 28 | 34 | 36 |
|---|---|---|---|---|---|
| | Symbol | | ∘ | ▭ | ⋄ |
| Polymer | Polymer Supplier/Name | | Evonik | Ashland | |
| | Co-Monomer Ratio | | 50:50 | 55:45 | |
| | Polymer IV (dL/g) | | 0.24 | 0.2 | |
| | Polymer Endcap | | Ester | | |
| Formulations | Batch Size (g) | | 5 | 500 | |
| | Mixing Speed (RPM) | | 3000 | | |
| | Target Drug Load (%) | | 50.0 | | |
| Analytical | Drug Load (%) | | 49.4 | 50.4 | 48.3 |
| | Encapsulation Efficiency (%) | | 98.8 | 100.8 | 96.7 |
| | Residual Solvent DCM (%) | | 0.2 | 0.4 | 0.0 |
| | Particle | Dᵥ10 | 12 | 18 | 16 |
| | Size | Dᵥ50 | 33 | 37 | 37 |
| | (μm) | Dᵥ90 | 57 | 63 | 62 |

| | | | |
|---|---|---|---|
| Sample MW (kDa) | 15.6 | 20.5 | 23.2 |
| Polymer MW (kDa) | 15.7 | 20.2 | 24.5 |

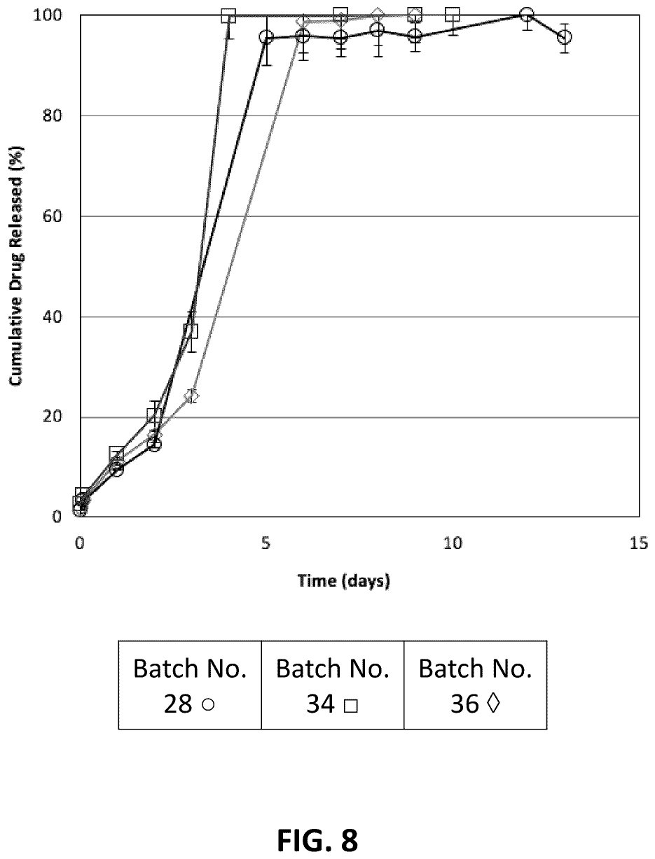

FIG. 8 is a graph showing in vitro cumulative ibrutinib release over time. Batch No. 36 was fully released in 6 days, similar to Batch No. 28, which released in 5 days. Batch No. 34 was fully released in 4 days, similar to Batch No. 28. The formulations have the same shape of release curve.

### Example 9—Scale Up and Optimization of Group D and Group D-Like Ibrutinib-Encapsulated Polymer Microspheres

Following the general procedure described in Example 1 and illustrated in FIG. 1, the DP was formed by dissolving 250 g of DLG 8503 A polymer, followed by addition of ibrutinib (250 g) with mixing until completely dissolved. The DP was filtered and pumped at a flow rate of 25 mL/min into a Levitronix® BPS-i100 integrated pump system operating at 3,000 RPM. The CP comprising 0.35% PVA was also pumped into the homogenizer at a flow rate of 2 L/min (CP:DP=80:1).

The formed or forming microspheres exited the homogenizer and entered the SRV. Deionized water was added to the SRV. Solvent removal was achieved using water washing and filtering. The bulk suspension was collected via filtration and lyophilized to obtain a free-flowing powder.

Batch No. 32 had a particle size of 34 μm (D₅₀), a drug load of 50.2 wt %, and a molecular weight of 22.6 kDa. The microspheres contained residual DCM of 0.7%. Batch No. 35 had a particle size of 36 μm (D₅₀), a drug load of 49.5 wt %, and a molecular weight of 26.0 kDa. The microspheres contained residual DCM of 0.4%. The parameters and results are shown tabularly in Table 8, relative to Batch No. 18:

TABLE 8

| Lot | Batch Number | | 18 | 32 | 35 |
|---|---|---|---|---|---|
| | Symbol | | Δ | ▭ | ⋄ |
| Polymer | Polymer Supplier/Name | | Ashland | | |
| | Co-Monomer Ratio | | 85:15 | | |
| | Polymer IV (dL/g) | | 0.24 | | |
| | Polymer Endcap | | Acid | | |
| Formulations | Batch Size (g) | | 5 | 500 | |
| | Mixing Speed (RPM) | | 3000 | | |
| | Target Drug Load (%) | | 50.0 | | |
| Analytical | Drug Load (%) | | 49.8 | 50.2 | 49.5 |
| | Encapsulation Efficiency (%) | | 99.6 | 100.4 | 98.9 |
| | Residual Solvent DCM (%) | | 0.5 | 0.6 | 0.4 |
| | Particle | Dᵥ10 | 15 | 14 | 17 |
| | Size | Dᵥ50 | 36 | 34 | 36 |
| | (μm) | Dᵥ90 | 62 | 58 | 61 |

| | | | |
|---|---|---|---|
| Sample MW (kDa) | 22.7 | 23.1 | 23.5 |
| Polymer MW (kDa) | 22.7 | 22.6 | 26.0 |

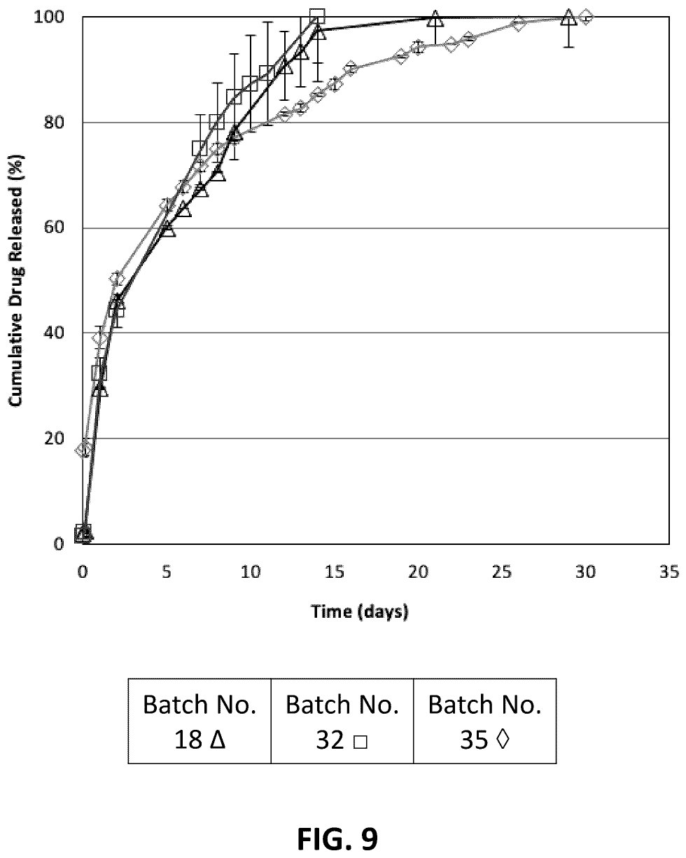

FIG. 9 is a graph showing in vitro cumulative ibrutinib release over time. Batch Nos. 18 and 25 released similarly between day 0-8. Batch No. 35 began to release slower after day 8 when compared to Batch No. 18, which was fully released by day 14. Batch No. 35 was 17% released at the first time-point.

In use, the microspheres may be suspended in a diluent for administration (injection). The diluent may generally contain a thickening agent, a tonicity agent, and a wetting agent. The thickening agent may include carboxymethyl cellulose-sodium (CMC-Na) or other suitable compounds. An appropriate viscosity grade and suitable concentration of CMC-Na may be selected so that the viscosity of the diluent is 3 cps or higher. Generally, a viscosity of about 10 cps is suitable; however, a higher viscosity diluent may be preferred for larger microspheres to minimize the settling of microspheres in the suspension.

Uniform microsphere suspension without particle settling will result in a consistent delivered dose during drug administration by injection. To have a tonicity of the diluent closer to the biological system, about 290 milliosmole (mOsm), solutes such as mannitol, sodium chloride, or any other acceptable salt may be used. The diluent may also contain a buffer salt to maintain the pH of the composition. Typically, the pH is maintained around a physiologically relevant pH by adjusting the buffer content as needed (pH about 7 to about 8).

The aspects disclosed herein are not intended to be exhaustive or to be limiting. A skilled artisan would acknowledge that other aspects or modifications to instant aspects can be made without departing from the spirit or scope of the invention. The aspects of the present disclosure, as generally described herein and illustrated in the figures, can be arranged, substituted, combined, separated, and designed in a wide variety of different configurations, all of which are contemplated herein.

Unless otherwise specified, “a,” “an,” “the,” “one or more of,” and “at least one” are used interchangeably. The singular forms “a”, “an,” and “the” are inclusive of their plural forms. The recitations of numerical ranges by endpoints include all numbers subsumed within that range (e.g., 1 to 5 includes 1, 1.5, 2, 2.75, 3, 3.80, 4, 5, etc.). The terms “comprising” and “including” are intended to be equivalent and open-ended. The phrase “consisting essentially of” means that the composition or method may include additional ingredients and/or steps, but only if the additional ingredients and/or steps do not materially alter the basic and novel characteristics of the claimed composition or method. The phrase “selected from the group consisting of” is meant to include mixtures of the listed group.

When reference is made to the term “each,” it is not meant to mean “each and every, without exception.” For example, if reference is made to microsphere formulation comprising polymer microspheres, and “each polymer microsphere” is said to have a particular ibrutinib content, if there are 10 polymer microspheres, and two or more of the polymer microspheres have the particular ibrutinib content, then that subset of two or more polymer microspheres is intended to meet the limitation.

The term “about” in conjunction with a number is simply shorthand and is intended to include ±10% of the number. That is, the number is intended to be read as if it was followed by the phrase, “±10%”. This is true whether “about” is modifying a stand-alone number or modifying a number at either or both ends of a range of numbers. In other words, “about 10” means from 9 to 11. Likewise, “about 10 to about 20” contemplates 9 to 22 and 11 to 18. In the absence of the term “about,” the exact number is intended. In other words, “10” means 10.

What is claimed is:

1. A microsphere formulation, comprising:
polymer microspheres, each polymer microsphere comprising:
(i) ibrutinib; and
(ii) a biodegradable polymer,
wherein each polymer microsphere comprises a drug load of the ibrutinib of greater than 30% by weight of the polymer microsphere, and
wherein the polymer microspheres have a particle size of less than 110 μm (D₅₀).

2. The microsphere formulation of claim 1, wherein the biodegradable polymer comprises a poly(D,L-lactide-co-glycolide).

3. The microsphere formulation of claim 1, wherein the biodegradable polymer comprises a poly(D,L-lactide-co-glycolide) having a lactide:glycolide ratio of 50:50.

4. The microsphere formulation of claim 3, wherein the biodegradable polymer is ester-terminated.

5. The microsphere formulation of claim 1, wherein the biodegradable polymer comprises a poly(D,L-lactide-co-glycolide) having a lactide:glycolide ratio of 55:45.

6. The microsphere formulation of claim 5, wherein the biodegradable polymer is ester-terminated.

7. The microsphere formulation of claim 1, wherein the biodegradable polymer comprises a poly(D,L-lactide-co-glycolide) having a lactide:glycolide ratio of 85:15.

8. The microsphere formulation of claim 7, wherein the biodegradable polymer is acid-terminated.

9. The microsphere formulation of claim 1, wherein the biodegradable polymer has an inherent viscosity between about 0.2 dL/g and about 0.24 dL/g.

10. The microsphere formulation of claim 1, wherein each polymer microsphere comprises a drug load of the ibrutinib of about 50% by weight of the polymer microsphere.

11. The microsphere formulation of claim 1, wherein the polymer microspheres have a particle size of between about 20 μm (D₅₀) and about 60 μm (D₅₀).

12. The microsphere formulation of claim 1, characterized in that about 75% to 100% of the ibrutinib is released over a period of about 12 to about 16 days of injection into a subject, but not more than about 20% of the ibrutinib has been released within 24 hours of injection into the subject.

13. The microsphere formulation of claim 1, characterized in that about 75% to 100% of the ibrutinib is released over a period of between about 25 to about 31 days of injection into a subject, but not more than about 20% of the ibrutinib has been released within 24 hours of injection into the subject.

14. A pharmaceutical composition comprising the microsphere formulation of claim 1.

15. The microsphere formulation of claim 1 for use in the treatment of a B-cell malignancy.

16. A microsphere formulation, comprising:
polymer microspheres, each polymer microsphere comprising:
(i) ibrutinib; and
(ii) a biodegradable polymer comprising an ester-terminated poly(D,L-lactide-co-glycolide) having a lactide:glycolide ratio selected from 50:50, 55:45, and 75:25, or a combination thereof;
wherein each polymer microsphere comprises a drug load of the ibrutinib of between about 30% and about 55% by weight of the polymer microsphere, and
wherein the polymer microspheres have a particle size of between about 25 μm (D₅₀) and 40 μm (D₅₀), and
wherein the microsphere formulation is characterized in that about 75% to 100% of the ibrutinib is released over a period of between about 12 to about 16 days of injection into a human subject, but not more than about 20% of the ibrutinib has been released within 24 hours of injection into the human subject.

17. A microsphere formulation, comprising:
polymer microspheres, each polymer microsphere comprising:
(i) ibrutinib; and
(ii) a biodegradable polymer comprising an acid-terminated poly(D,L-lactide-co-glycolide) having a lactide:glycolide ratio of 85:15;
wherein each polymer microsphere comprises a drug load of the ibrutinib of between about 45% and about 55% by weight of the polymer microsphere, and
wherein the polymer microspheres have a particle size of between about 30 μm (D₅₀) and 40 μm (D₅₀), and
wherein the microsphere formulation is characterized in that about 75% to 100% of the ibrutinib is released over a period of between about 25 to about 31 days of injection into a human subject, but not more than about 20% of the ibrutinib has been released within 24 hours of injection into the human subject.
